(19) United States
(12) Patent Application Publication
(10) Pub. No.: US 2006/0084969 A1
(43) Pub. Date: Apr. 20, 2006

(54) Endovascular catheter and method of use

(76) Inventors: Csaba Truckai, Saratoga, CA (US); John H. Shadduck, Menlo Park, CA (US)

Correspondence Address: Attn: John H. Shadduck;SurgRx Inc., 380 Portage Ave, Palo Alto, CA, 94306, US

(21) Appl. No.: 11/199,580

(22) Filed: Aug. 8, 2005

Related U.S. Application Data
(63) Continuation-in-part of application No. 11/118,881, filed on Apr. 29, 2005.
(63) Continuation-in-part of application No. 10/351,449, filed on Jan. 22, 2003.
(63) Continuation-in-part of application No. 10/643,787, filed on Aug. 19, 2003.
(60) Provisional application No. 60/601,497, filed on Aug. 14, 2004.
(60) Provisional application No. 60/602,500, filed on Aug. 17, 2004.

(51) Int. Cl. A61B 18/14

(52) U.S. Cl. 606/41; 606/52

(57) ABSTRACT

An electrosurgical medical device and method for treating cardiac arrhythmias. In one embodiment, an elongate catheter has a distal shaft end or balloon that carries an electrosurgical energy delivery surface comprising at least one electrode with a positive temperature coefficient of resistance (PTCR) surface and/or an electrode with a pressure sensitive variable resistance to provide a smart surface for controlling Rf current flow at the interface of electrosurgical surface and the tissue. The electrosurgical surface then can limit or modulate Rf energy delivery through the surface in response to the temperature of the tissue or the engagement pressure of the surface against the engaged tissue. In operation, the smart electrosurgical surface can prevent arcing at the electrode-tissue interface, and thus control ohmic heating to prevent tissue desiccation, charring and emboli formation.

### CROSS-REFERENCE TO RELATED APPLICATIONS

This application claims benefit the following Provisional U.S. patent applications: Ser. No. 60/601,497 filed Aug. 14, 2004 titled Endovascular Catheter and Method of Use and Ser. No. 60/602,500 filed Aug. 17, 2004 titled Endovascular Catheter and Method of Use. This application is a continuation-in-part of co-pending U.S. patent application Ser. No. 10/032,867 filed Oct. 22, 2001; and is a continuation-in-part of U.S. patent application Ser. No. 10/351,449 filed Jan. 22, 2003; and is a continuation-in-part of U.S. patent application Ser. No. 10/441,519 filed May 20, 2002; and is a continuation-in-part of U.S. patent application Ser. No. 10/781,925 filed Feb. 14, 2004. All of the above applications are incorporated herein by this reference and made a part of this specification.

### BACKGROUND OF THE INVENTION

1. Field of the Invention

The present invention relates to electrosurgical working ends and catheters for treatment of cardiac arrhythmias. In a particular embodiment, the invention relates to a catheter working end for delivering Rf current to tissue through a polymeric PTCR surface material and/or a pressure sensitive variably resistive material for controllably creating lesions in a patient's left atrium to electrically isolate a pulmonary vein from the upper chamber of the heart.

2. Description of Related Art

Cardiac arrhythmias or irregular heartbeats are a common disorder. The heart muscle carries an electrical system which is responsible for causing it to contract and relax to provide a normal rhythm. Normally, each heartbeat begins in the SA node that comprises a group of cells located in the upper right chamber of the heart (cf. FIG. 1). An electrical impulse travels from the SA node and spreads across both upper chambers to cause the atria to contract. The electrical impulse next travels to the AV node, located between the upper and lower chambers of the heart. Thereafter, the electrical impulse leaves the AV node and spreads down and across both lower chambers (the ventricles). The ventricles contract thus pumping blood to the body.

Supraventricular tachycardia (SVT) is a general term describing any arrhythmia, or abnormal heart rhythm, originating above the ventricles. Specific types of SVT include atrial fibrillation, AV nodal re-entrant tachycardia, and Wolff-Parkinson-White syndrome. For example, in Wolf-Parkinson-White (WPW) syndrome, there exists an anomalous A-V conduction pathway, causing retrograde conduction from the ventricles to the atria. This extra electrical pathway between the atria and ventricles at times causes a rapid rhythm. Instead of allowing the next heartbeat to begin at the SA node, the abnormal conduction pathway can pick up an electrical impulse in the ventricles and send it abnormally back upward to the atria. When this happens, the impulse begins to travel abnormally in a rapid, circular manner, causing a rapid heart rate. This irregular conduction pathway is caused by an extraneous strand of muscle fiber in the heart wall that can short-circuit the normal electrical impulses within the heart. In AV nodal re-entrant tachycardia, the AV node has two or more groups of conductive cells. Because of the extra conduction pathways, the heart can at times beat more rapidly than normal. Atrial fibrillation (AF) is an arrhythmia causes by rapid electrical impulses coming from the atria (the upper chambers of the heart) that are disorganized and irregular.

A radiofrequency ablation procedure (RFA) is a standard treatment for several forms of abnormal heart rhythms. Techniques have been developed for mapping the locations of tachycardias to thereby target particular abnormal tissues. A RFA procedure is then used to disable the “functional” short-circuits in tissue. Typically, catheter-based radiofrequency (Rf) energy is applied to a particular location or region to produce a lesion or scar that can interrupt electrical conduction through the treated region. Such high frequency electrical energy also can be used to “disconnect” the electrical pathway between the upper chambers (atria) and the lower chambers (ventricles) of the heart. Whether a particular type of RFA treatment is suitable depends upon the type of arrhythmia. Patients with AF also can be candidates for an open surgical intervention called the Maze procedure. A series of precise elongate incisions (resulting in elongate scars) are made in both the right and left atria in an attempt to confine the electrical impulses to defined pathways. Although the number of patients who have undergone this procedure is relatively small, many have achieved long-term freedom from AF. Clinical investigations are ongoing to determine if RFA procedures can be developed to duplicate the Maze procedure.

Instruments and techniques are needed (i) that create endocardial lesions with elongate, arcuate or circular patterns that are precisely localizable relative to mapped target locations, (ii) that create lesions that extend a selected depth within a cardiac wall or vein wall; (iii) that create lesions without charring surface tissue as common in radiofrequency ablation procedures; and (iv) that can create lesions that have more defined edge conditions between the lesion perimeter and unscarred tissue by elimination of stray electrical current flow common in radiofrequency ablation procedures.

### SUMMARY OF THE INVENTION

In general, the apparatus of the invention comprises a catheter device with an electrosurgical working end that can be articulated for creating elongate, arcuate lesions in endocardial tissue to electrically isolate pulmonary veins from the left atrium. In one embodiment, a central wire element can be axially retracted to create a loop-like region of an electrosurgical energy-delivery surface. In another embodiment, the working end carries a balloon structure. Of particular interest, the working end or balloon carries an electrosurgical surface of a positive temperature coefficient of resistance (PTCR) polymer that can limit Rf current and energy density in cardiac tissue without thermocouples and controller feedback circuitry. The PTCR polymer consists of first and second portions—preferably including an elastomer such as silicone (first portion) and conductive particles (second portion) distributed therein. Either mono-polar or bi-polar Rf energy delivery is possible. The PTCR electrosurgical surface exhibits unique resistance vs. temperature characteristics with a low base resistance over a selected temperature range and a dramatically increasing resistance above a selected narrow temperature range as the PTCR material senses ohmic heating in tissue.

In operation, it can be understood that current flow through the PTCR surface will cause active Rf energy (ohmic heating) in the engaged tissue until the point in time that any portion of the matrix is heated to the predetermined range that substantially reduces the conductance of the PTCR material. This effect will occur across the surface of the PTCR material thus allowing each PTCR portion to deliver an independent level of power therethrough. This instant, localized reduction of Rf energy application can be relied on to prevent any dehydration and desiccation of tissue proximate to the electrosurgical surface. The system eliminates the possibility of tissue charring and the potential of emboli. In operation, the working end can modulate the application of Rf energy to the engaged tissue to create a lesion having a predetermined depth and shape.

In another embodiment, the catheter of the present invention has a working end that defines a tissue-contacting surface with a plurality of media entrance ports. A fluid media source is fluidly coupled to the media entrance ports by a fluid channel. Fluid inflow, such as a saline solution, can be ejected from the PTCR surface contemporaneous with Rf energy application.

In another embodiment, the catheter working end carries an electrosurgical surface comprising a pressure sensitive variably resistive material for controlling or limiting Rf current flows in tissue based on engagement pressure. Such a pressure sensitive material also can assist in preventing arcing and tissue desiccation.

The instrument and method of the invention advantageously can create arcuate or circular lesions in endocardial tissues.

The instrument and method of the invention creates lesions in targeted tissue without causing tissue desiccation or surface carbonization common to electrosurgical instruments and techniques.

The instrument and method of the invention can treat endocardial tissues without tissue charring, thus preventing any possibility that charred material will be migrate and cause the risks, such as in embolic stroke.

The instrument and method advantageously can create a lesion in endocardial tissues for blocking conduction pathways that is substantially narrow and substantially deep within cardiac tissue.

The instrument and method advantageously can creates a lesion in endocardial tissues for blocking conduction pathways that comprises an elongate linear lesion or comprises a spot lesion.

Additional advantages of the invention will be apparent from the following description, the accompanying drawings and the appended claims.

### BRIEF DESCRIPTION OF THE DRAWINGS

FIG. 1 is a cut-away view of a patient's heart with the catheter working end guided to the left atrium.

FIG. 2A is a side view of the Type “A” catheter of FIG. 1 in a first linear position or shape for guiding over a guidewire, the working end having a PTCR electrosurgical surface for causing controlled ohmic heating of engaged tissue.

FIG. 2B is a side view of the Type “A” catheter of FIG. 2B in a second expanded shape for creating a circular lesion about the entrance of a pulmonary vein into the left atria.

FIG. 3 is a cut-away view of a patient's heart as in FIG. 1 with the catheter working end articulated and wherein the PTCR surface creates an arcuate lesion pattern around a pulmonary vein.

FIG. 4 is a plan view of an alternative Type “B” catheter working end with a balloon having a PTCR surface that is adapted to create a lesion within or about a pulmonary vein.

FIG. 5A is a view of an alternative balloon having a PTCR surface that is adapted to create an elongate lesion pattern within or about a pulmonary vein.

FIG. 5B is a view of an alternative balloon having a PTCR surface that is adapted to deliver ohmic heating within a wide region of a blood vessel.

FIG. 6 depicts a method of the invention wherein a balloon PTCR surface causes controlled ohmic heating to create an annular lesion in a blood vessel.

FIG. 7 is a view of an alternative balloon catheter that carries a PTCR surface coupled to an Rf source.

FIG. 8 is a view of another balloon catheter wherein the electrosurgical energy delivery surface is a pressure sensitive variably resistive material.

FIG. 9 is a view of clamp device with first and second jaws with a PTCR electrosurgical energy delivery surface and/or a pressure sensitive variably resistive electrosurgical surface in a jaw.

### DETAILED DESCRIPTION OF THE INVENTION

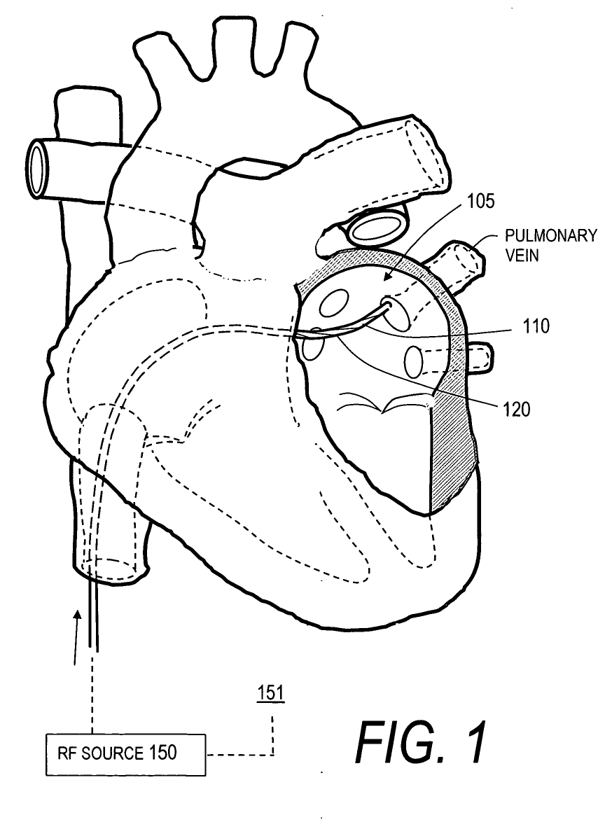

1. Type “A” catheter with PTCR electrosurgical surface. FIG. 1 illustrates a Type “A” electrosurgical catheter 100 with a distal working end region 105 having a PTCR electrosurgical surface 110 corresponding to the invention after being guided over a guidewire to the left atrium 112. The scope of the invention extends to any endoluminal catheter that includes any electrical conductor coupled to a voltage source that has such a PTCR surface for controlling Rf current flow and the resultant ohmic heating of engaged tissue. The cross-section of the catheter sleeve 100 can be a suitable dimension, for example, from 2 to 10 French OD. The catheter can have a bore therein dimensioned to slide over a guidewire.

The PTCR electrosurgical surface 110 is a polymeric composition that is doped with conductive particles. The PTCR composition is described in more detail in the co-pending patents listed in the Section above titled CROSS-REFERENCE TO RELATED APPLICATIONS. In one embodiment, the PTCR surface is conductively coupled to an Rf source and operates in a mono-polar manner in cooperation with a ground pad. It can be easily understood that bi-polar systems are possible with spaced apart first and second opposing polarity PTCR regions within the working end.

Describing a material as having a positive temperature coefficient of resistance (PTCR or PTC) simply means that the resistance of the material increases as temperature increases. Practically any material that exhibits metal-like conduction has a positive temperature coefficient of resistance. (Materials that conduct like metals have the lowest resistivity of all non-superconducting materials, wherein resistivity generally falls in the range of 1-100 μΩ-cm.). In these metal-like materials, the PTCR variable resistivity effect is characterized by a gradual increase in resistance that is linearly proportional to temperature—that is, a linear PTCR effect.

A “nonlinear” PTCR effect is exhibited by certain types of polymer matrices that are doped with conductive particles. In general, these polymer PTCR materials comprise a non-conductive base polymer that exhibits two phases (at a phase change or glass transition temperature T_{G}) that define greater and lesser conductive states. The first phase is a crystalline or semi-crystalline state where the polymer molecules form long chains and are arranged in a more ordered architecture. When the temperature of the material is elevated, the polymer molecules maintain the crystalline architecture or structure through a selected temperature range. The polymer is designed to transition to an at least partly amorphous phase from the crystalline state at a selected temperature range. In the amorphous state, the molecules are aligned more randomly, and there may be slight changes in material geometry at the macroscale. The non-conductive polymer is combined with a dispersed, highly conductive particles (e.g., carbon micro- or nanoparticles) to form a matrix. In the crystalline phase of the polymer, the carbon particles are packed into the crystalline boundaries and form many conductive paths across and through the matrix material. In this low temperature crystalline state, the polymer-carbon matrix is engineered to have a low resistance.

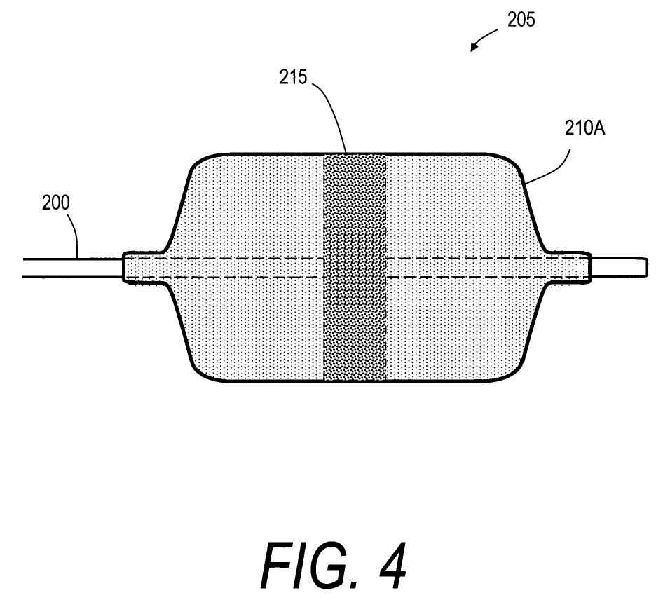

For the purposes of the present invention, the PTCR surface in a catheter body (see FIGS. 1-3) or in a balloon wall (see FIG. 4) is adapted to contact tissue and allow conduction of Rf energy therethrough to the engaged tissue T to cause ohmic tissue heating. After the tissue is elevated in temperature, heat is conducted from the engaged tissue back to the PTCR surface or body to thereby elevate the temperatures in at least surfaces regions of the polymer.

As long as the temperature increase in the matrix portion adjacent the ohmically heated tissue does not cause a phase change in the polymer, current can flow unimpeded through the PTCR surface. When the temperature of the PTCR surface is elevated to a selected temperature, called a switching range herein, the temperature will cause a phase change in the polymer. The crystalline structure of the polymer will disappear, the polymer volume will expand, and the carbon chains that allow conduction across the matrix will be broken—resulting in an extraordinary increase in resistance. The polymer-carbon matrix can define a resistance measured in milliohms or ohms before the phase change. After the phase change, the matrix′ resistance can be measured in megaohms. Current flow can be reduced accordingly, or terminated, which is used in particular manners corresponding to the invention to precisely control Rf energy densities in the engaged tissue.

The process described above is reversible so that when an affected portion of a PTCR surface falls in temperature, the polymer component will return to its crystalline structure and that particular matrix volume will return its original state. The conductive carbon particles will reform into conductive paths within the interstices of the crystalline polymer architecture.

As the temperature of the matrix falls, it appears that the exact same conductive paths may not exactly reform themselves after first use of the matrix, and for this reason the polymer matrices of the invention may be temperature cycled several times in the fabrication process which appears to cause the material to have substantially resettable conductive paths. In the fabrication process, the PTCR surface also can be treated in various processes (e.g., gamma, UV irradiation etc.) to cross-link the polymer or co-polymers of the matrix.

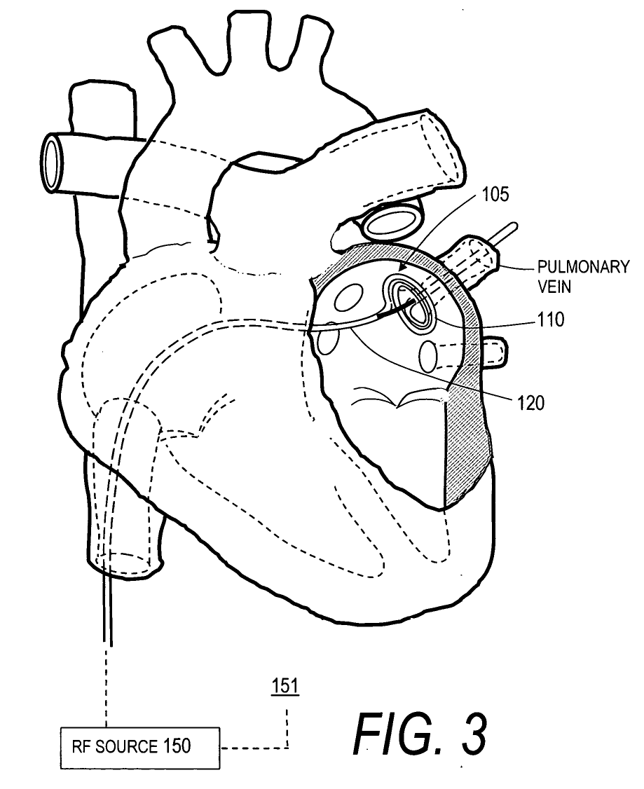

In an exemplary embodiment, the PTCR surface can be designed to have a temperature-impedance curve that maintains a relatively low base resistance over a selected base temperature range with a dramatically increases resistance above a selected narrow temperature range (switching range) that can be any 1° to 10° range between about 50° C. and 200° C., and more preferably between about 70° C. and 90° C. In use, the PTCR surface of FIGS. 3 and 4 can self-modulate current flow among multiple paths through the engaged tissue depending on the temperature of surface regions of the engaged tissue and the conduction parameters of such tissue regions.

The PTCR material or surface can comprise any suitable polymer as described above with the dispersed conductive filler or dopant being any suitable particle or filament, e.g., silver, tin, nickel, gold, copper, platinum, palladium, magnesium, aluminum, molybdenum, tungsten, tantalum, zinc, cobalt, carbon or combinations thereof. The conductive filler material can comprise between about 20% and 80% by volume of the energy emitter material.

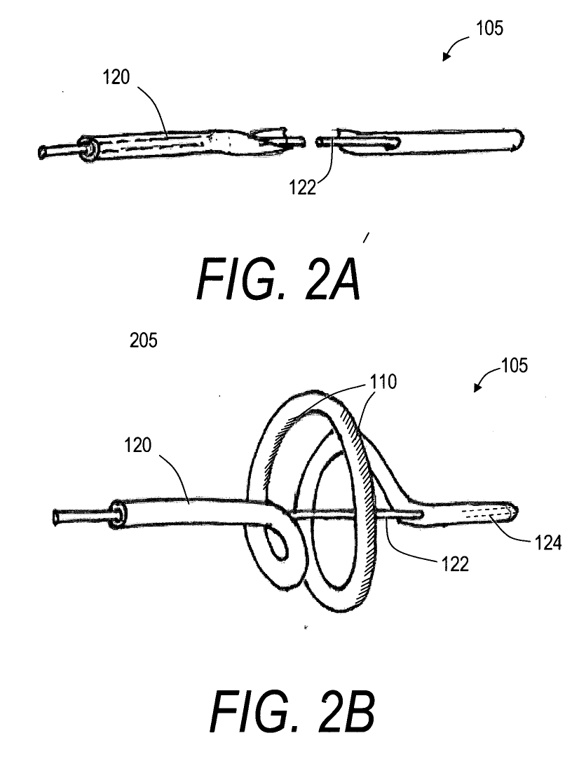

In one embodiment, FIGS. 2A and 2B illustrate the distal working end 105 of the electrosurgical catheter of FIG. 1 that is adapted for articulation to form an arcuate or circular lesion around the entrance of a pulmonary vein in the left atria. In FIG. 2A, the working end 105 has a deformable portion 120 that is disposed helically in relation to a central pull-cable 122. The central pull-cable 122 can have a bore 124 therein for a guidewire. It can be seen in FIG. 2B that axially movement of the central pull-cable 122 will articulate the deformable portion 120 and allow its reconfiguration to an arcuate shape. In one embodiment, the selected articulated configuration is a semi-circular shape, of a shape with an open center. In a preferred embodiment, the selected articulated configuration is a spiral shape, a loop shape that extends at 360°about the catheter axis.

FIG. 3 illustrates how the distal working end 105 and spiral portion can be pressed against the wall of the atrium to form a circular lesion pattern. The distal most end of the catheter can have a balloon for expanding in pulmonary vein (not shown) to stabilize the catheter and allow axial force to be applied to the spiral catheter portion.

In another embodiment, the PTCR surface 110 is porous and in fluid communication with saline source. Saline inflow in an about the surface may enhance ohmic heating. In another embodiment, the PTCR surface 110 is in a tubular sleeve that is wrapped about the catheter shaft. The tubular sleeve can be deflated for wrapping around the catheter to allow a smaller cross section working end in a pre-deployed condition. The tubular sleeve can inflated in cross-section to provide a deployed configuration.

The PTCR matrix can have a base polymer of a silicone or polyethelene or another thermosetting polymer such as a polyester as or a polypropylene. Some suitable materials for the base polymer of the matrix include, besides silicone, a polytetrafluoroethylene (PTFE) or a polyperfluoroalkoethylene such as a DuPont Kalrez® Compound, an Ausimont Hyflon® Fluoropolymer, or a Dupont Teflon® material. The conductive particles can be carbon, gold, platinum, silver, or a stainless steel coated with gold, platinum, silver or the like. In one embodiment, the ration by weight of the polymer-to-conductive particles can range from about 10/90 to about 70/30 (polymer/carbon particles) to provide the selected range at which the sleeve wall will function to substantially limit electrical conductance therethrough. More preferably, the percentage of carbon particles in an exemplary PTCR material is from about 40% to 80% with the balance being polymer.

Of particular interest, the PTCR surface illustrated in FIG. 3 will function to limit Rf current flows from Rf source 150 and the resultant ohmic heating in the engaged tissue of the atrial wall. This effect will occur across the engagement surface layer to provide spatially localized modulation of Rf current flows and energy density in the engaged tissue. In a mono-polar method in cooperation with ground pad 151, the PTCR material will sense the tissue temperature due to ohmic heating therein and thereby increase in resistance to limit ohmic heating to the tissue proximate to the PTCR surface 110.

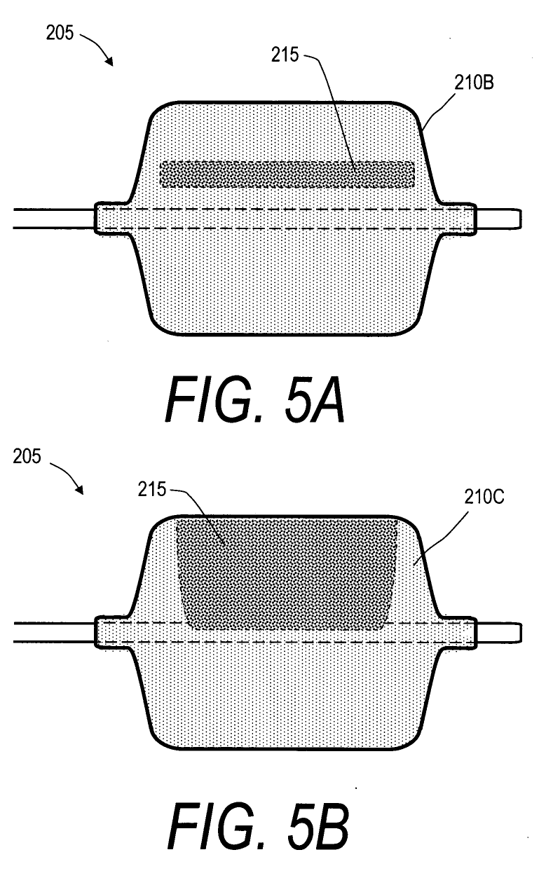

FIG. 4 illustrates an alternative catheter 200 with working end 205 that includes an expandable balloon 210A for engaging the walls of a vessel lumen or about the atrium of a patient's heart. A number of endovascular procedures can benefit from a balloon having a PTCR surface or surface portion indicated at 215. For convenience, an exemplary embodiment of balloon 210A will be described in more detail that is adapted for treating cardiac arrhythmia such as atrial fibrillation. In one treatment of atrial fibrillation, the objective is to create a circumferential lesion about a pulmonary vein adjacent the heart to block conduction pathways. FIG. 4 illustrates a balloon 210A that is designed for creating such a circumferential lesion with PTCR emitter band 215 extending 360° around a central region of the balloon. The use of mono-polar and bi-polar thermal energy emitters will be described in more detail below. It should be appreciated that the scope of the invention relating to polymer balloon walls extends to any geometry of PTCR surface portion for modulating ohmic heating of tissue, for example as illustrated in FIGS. 5A and 5B. FIG. 5A depicts a balloon 210B having at least one axially-extending PTCR surface 215 that may be used to create elongate lesions in a heart wall to treat atrial fibrillation. FIG. 5B illustrates balloon 210C with a broad PTCR portion 215. In one embodiment, the balloon 210C of FIG. 5B is adapted for thermal treatment of a wider area of a vessel wall, for example, to treat a location of vulnerable plaque wherein very controlled heat can polymerize the lipid pool within the vessel wall to prevent its rupture.

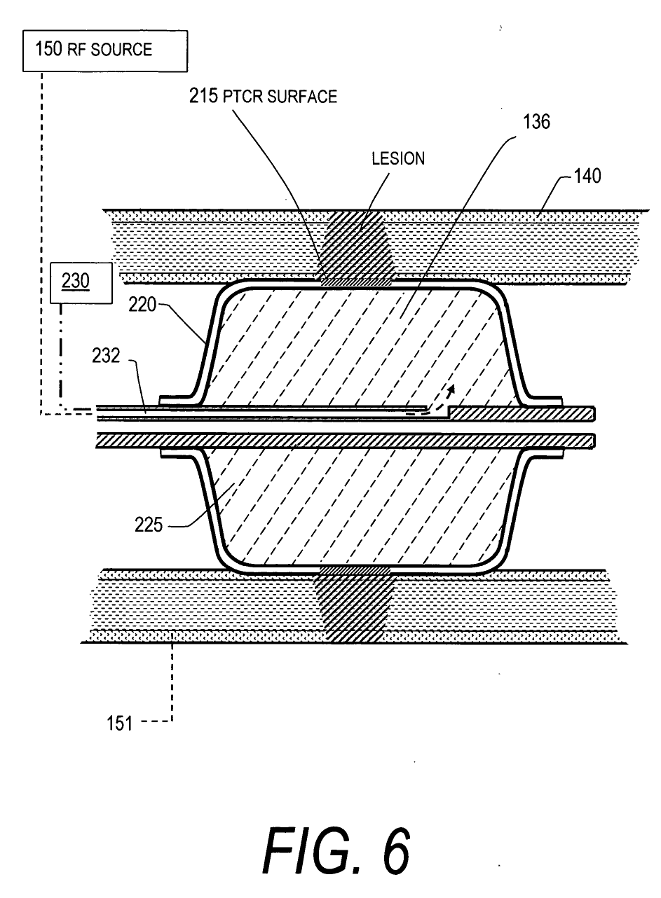

In sectional views of the balloon wall 220 of FIG. 6, the polymeric material comprising wall 220 is any crystalline or semi-crystalline polymer that is known in the art of catheter balloons, and can be PET (polyethylene terephthalate), nylon, polyolefin or other polyethylene. The polymers described above are well known and are available from Dow Chemical, Union Carbide or Dupont-Mitsui Polychemicals Co., Ltd., all of which manufacture one or more of the above polymers.

Still referring to FIG. 6, the balloon is similar to that of FIG. shown in use wherein the PTCR surface 215 comprises a polymeric element that is sealably deposited, bonded or otherwise adhered to the balloon wall. The balloon 200 is of a non-distensible polymer to provide satisfactory coupling of the layer PTCR surface 215 and the balloon surface. In one embodiment, the PTCR surface 215 overlies an electrode film 221 that is coupled to Rf source 150. In another embodiment, the system can utilize a conductive fluid media 225 as an electrical conductor to allow the PTCR component 215 to function as a mono-polar electrode, which can reduce the complexities of manufacturing the catheter working end. In this embodiment, the PTCR material extends through the wall 220 of balloon 200. In FIG. 6, a fluid source such as a syringe communicates with inflow channel 232 to expand the balloon 200. The fluid media 225 is a highly conductive saline solution that contacts the electrode PTCR portion 215. An electrode of any shape and configuration is included in the working end shaft or in the interior of channel 234 or within the balloon interior chamber 236. In this embodiment, there is no need to fabricate circuitry in the balloon wall to couple with a film electrode 221. In FIG. 6, the thermal effects of mono-polar Rf energy as known in the art are illustrated with lesion 244 extending around a pulmonary vein. The mono-polar PTCR electrosurgical surface cooperates with ground pad 151.

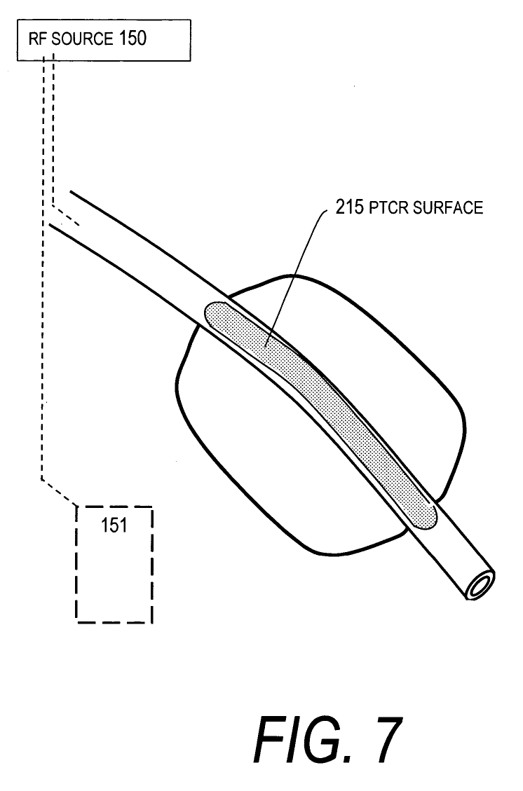

FIG. 7 illustrates an alternative catheter working end that falls within the scope of the invention wherein a balloon can be used to press a PTCR surface 215 of a catheter working end against tissue to create an elongate lesion. In all other respects, the PTCR electrosurgical surface 215 functions as described above.

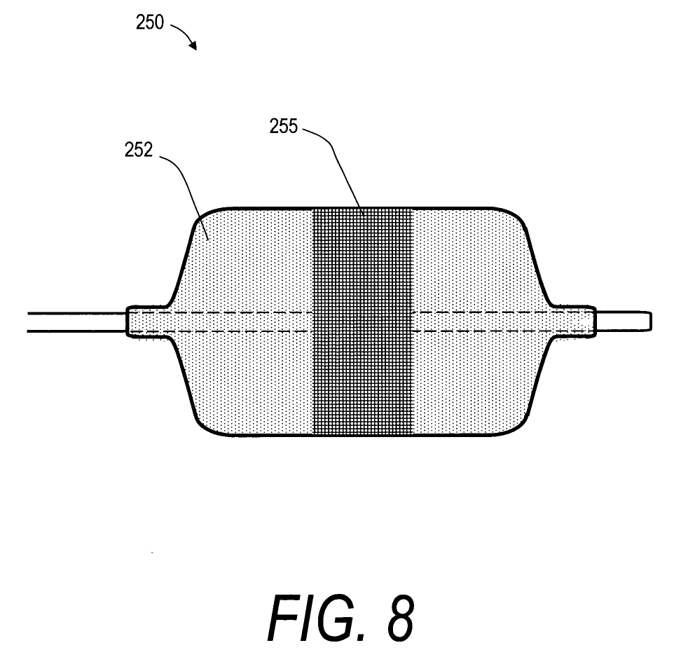

FIG. 8 illustrates an alternative catheter working end 250 that falls within the scope of the invention wherein a balloon 252 has an expandable wall that includes a pressure sensitive variably resistive polymer portion 255. Pressure sensitive variably resistive materials are conductively doped elastomers that bear a resemblance to PTCR materials described above. Some materials suitable for any embodiments of the invention exhibit both PTCR characteristics and pressure sensitive variably resistance. Some pressure sensitive materials are commercially available from Peratech Ltd, G3/G4 Morton Park Way, Darlington Co, Durham, England DL14PJ. A method of the invention comprises engaging cardiac tissue with a pressure sensitive variable resistance Rf electrosurgical surface, and delivering bi-polar or mono-polar Rf current from the delivery surface to ohmically heat the cardiac tissue. During use, the pressure sensitive variable resistance material allows Rf current flow only to tissue adjacent regions of said material that is under a selected level of engagement pressure, which can prevent arcing and eschar about the interface Rf electrosurgical surface and the tissue.

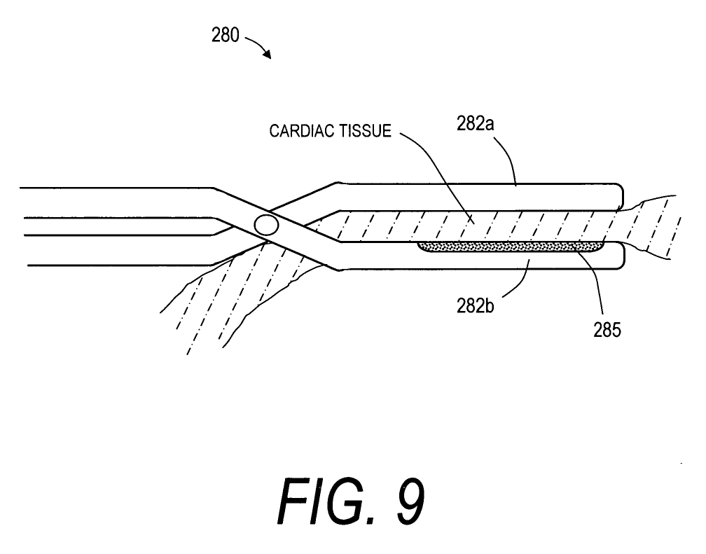

FIG. 9 illustrates an alternative instrument comprising a clamp structure 280 for engaging cardiac tissue as in known in the art in an open or endoscopic procedure wherein a first jaw 282a with a PTCR electrosurgical surface 285 is inserted through an incision in the heart to engaged endocardial tissue. The second jaw 282b is positioned on the exterior of the heart wall to clamp tissue. Thereafter, the PTCR electrosurgical surface 285 is activated to deliver mono-polar or bi-polar energy to the tissue to create a lesion. The PTCR electrosurgical surface 285 functions as described above to prevent arcing, tissue desiccation and carbonization.

Although particular embodiments of the present invention have been described above in detail, it will be understood that this description is merely for purposes of illustration. Specific features of the invention are shown in some drawings and not in others, and this is for convenience only and any feature may be combined with another in accordance with the invention. Further variations will be apparent to one skilled in the art in light of this disclosure and are intended to fall within the scope of the appended claims.

1-24. (canceled)

25. A method for treating cardiac arrhythmia, the method comprising:
engaging cardiac tissue with an electrosurgical surface including an electrode with a coating of a pressure sensitive variable resistance material; and
delivering Rf current from the electrosurgical surface thereby ohmically heating the cardiac tissue, wherein the pressure sensitive variable resistance material allows Rf current flow only to cardiac tissue adjacent regions of said material that is under a selected level of engagement pressure.

26. The method of claim 25 wherein the engaging step includes guiding a catheter working end carrying the electrosurgical surface to an endocardial location.

27. The method of claim 26 wherein the engaging step includes increasing a cross-sectional dimension of the working end.

28. The method of claim 26 wherein the engaging step includes articulating at least a portion of the working end.

29. The method of claim 26 wherein the engaging step includes moving the working end to an arcuate shape.

30. The method of claim 26 wherein the engaging step includes clamping cardiac tissue.

31. The method of claim 26 wherein the engaging step includes engaging tissue about the entrance to a pulmonary vessel.

32. The method of claim 26 wherein delivering Rf current occurs between the electrosurgical surface and a ground pad.

33. The method of claim 26 wherein delivering Rf current occurs between opposing polarity portions of the electrosurgical surface.

34. The method of claim 27 wherein increasing the cross-sectional dimension of the working end is carried out by axially moving a pull-wire.

35. The method of claim 27 wherein increasing the cross-sectional dimension of the working end provides at least one of a spiral shape, a semi-circular shape or loop shape.

36. The method of claim 27 wherein increasing the cross-sectional dimension of the working end is carried out by expanding a balloon member.

37. A method for treating cardiac arrhythmia, the method comprising:
engaging cardiac tissue with an electrosurgical surface including an including a positive temperature coefficient of resistance (PTCR) material;
delivering Rf current from the electrosurgical surface thereby ohmically heating the cardiac tissue, wherein the Rf current flows are limited by changes in temperature in at least portions of the PTCR material to preventing arcing.

38. A device for treating cardiac arrhythmia comprising a probe having a working end configured for engaging cardiac tissue, the working having an electrosurgical surface including at least one of a material having a pressure sensitive variable resistance and having a positive temperature coefficient of resistance (PTCR), and an electrical source operatively coupled to the electrosurgical surface.

39. The device for treating cardiac arrhythmia of claim 38 wherein the electrosurgical surface is a surface portion of an expandable balloon.

40. The device for treating cardiac arrhythmia of claim 39 wherein at least a portion of balloon is porous.

41. The device for treating cardiac arrhythmia of claim 40 further comprising a saline source in fluid communication with an interior chamber of the balloon.

42. The device for treating cardiac arrhythmia of claim 38 wherein the electrosurgical surface has a first polarity for cooperating with a ground pad.

43. The device for treating cardiac arrhythmia of claim 38 wherein the electrosurgical surface has a first polarity portion and a second polarity portion.

44. The device for treating cardiac arrhythmia of claim 38 wherein the electrosurgical surface is in at least one tissue-engaging surface of an openable-closeable jaws structure.
